(19) United States
(12) Patent Application Publication
(10) Pub. No.: US 2019/0256454 A1
(43) Pub. Date: Aug. 22, 2019

(54) NEW PROCESS FOR EARLY SACUBITRIL INTERMEDIATES

(71) Applicant: Novartis AG, Basel (CH)

(72) Inventors: Florian Karl KLEINBECK-RINIKER, Zurich (CH); Tobias KAPFERER, Basel (CH)

(21) Appl. No.: 16/314,933

(22) PCT Filed: Jun. 30, 2017
(86) PCT No.: PCT/IB2017/053971

§ 371 Date: Jan. 3, 2019

(30) Foreign Application Priority Data
Jul. 5, 2016 (EP) 16178030.9

(51) Int. Cl. C07C 215/28; C07C 229/10; C07C 225/16

(52) U.S. Cl. CPC C07C 215/28; C07D 265/28; C07C 225/16; C07C 229/10

(57) ABSTRACT

The invention relates to a new enantioselective process for producing useful intermediates for the manufacture of NEP inhibitors or prodrugs thereof, in particular NEP inhibitors comprising a γ-amino-δ-biphenyl-α-methylalkanoic acid, or acid ester, backbone.

### FIELD OF THE INVENTION

The present invention relates to a new chemical synthesis route for intermediates useful for the preparation of neprilysin (NEP) inhibitors and their prodrugs, in particular for the NEP inhibitor prodrug sacubitril.

### BACKGROUND OF THE INVENTION

The NEP inhibitor prodrug sacubitril (N-(3-carboxyl-1-oxopropyl)-(4S)-(p-phenylphenylmethyl)-4-amino-(2R)-methyl butanoic acid ethyl ester; IUPAC name 4-{[(1S,3R)-1-([1,1′-biphenyl]-4-ylmethyl)-4-ethoxy-3-methyl-4-oxobutyl]amino}-4-oxobutanoic acid, also known as AHU377) is represented by the following formula (A)

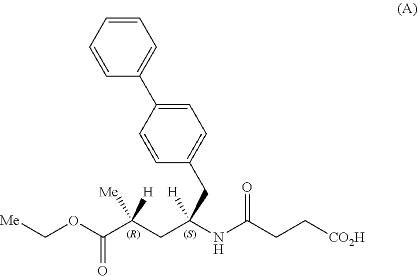

Sacubitril together with valsartan, a known angiotensin receptor blocker (ARB), forms a sodium salt hydrate complex, known as LCZ696, comprising the anionic forms of sacubitril and valsartan, sodium cations and water molecules in the molar ratio of 1:1:3:2.5, respectively (ratio of 6:6:18:15 in the asymmetric unit cell of the solid state crystal) (WO 2007/056546), and which is schematically present in formula (B).

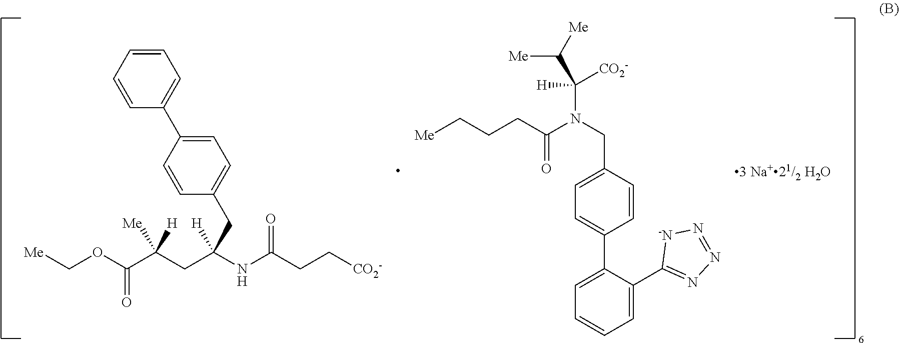

Said complex is also referred to by the following chemical names: trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate or octadecasodium hexakis(4-{[(1 S,3R)-1-([1,1′-biphenyl]-4-ylmethyl)-4-ethoxy-3-methyl-4-oxobutyl]amino}-4-oxobutanoate) hexakis(N-pentanoyl-N-{[2′-(1H-tetrazol-1-id-5-yl)[1,1′-biphenyl]-4-yl]methyl}-L-valinate)-water (1/15) (IUPAC nomenclature).

LCZ696 acts as angiotensin receptor neprilysin inhibitor (ARNI) and is therefore useful particularly in the treatment of hypertension or chronic heart failure. Its utility has been confirmed by clinical trials, e.g. in the landmark PARADIGM-HF trial.

Chemical synthesis routes to prepare NEP inhibitors and their prodrugs, in particular sacubitril, and its precursors have been described previously, e.g. in Ksander et al. J. Med. Chem. 1995, 38, 1689-1700; in U.S. Pat. No. 5,217,996 and in the international patent applications WO 2007/083774, WO 2007/083776, WO 2008/031567, WO 2008/083967, WO 2008/120567 WO 2009/090251, WO 2010/081410, WO 2011/035569, WO 2011/088797, WO 2012/025501, WO 2012/025502, WO 2013/026773, WO 2014/032627 and WO 2015/024991, as well as in CN patent applications CN102260177, CN103483201, CN104557600, CN104725256, CN104725279, CN105017082, CN105061263, CN105085322, CN105152980, CN105168205, CN105198775, CN105237560, CN105330569, CN105481622, CN105566194 and CN105601524 and others.

In particular WO2013/026773, WO2014/032627 and WO2015/024991 deal with novel synthesis methods to provide the precursor compound

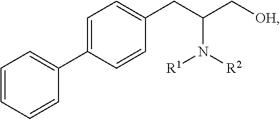

in particular

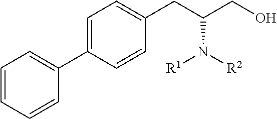

wherein R1 and R2 are independently of each other hydrogen or a nitrogen protecting group.

The process disclosed in WO 2013/026773 is depicted in the following scheme

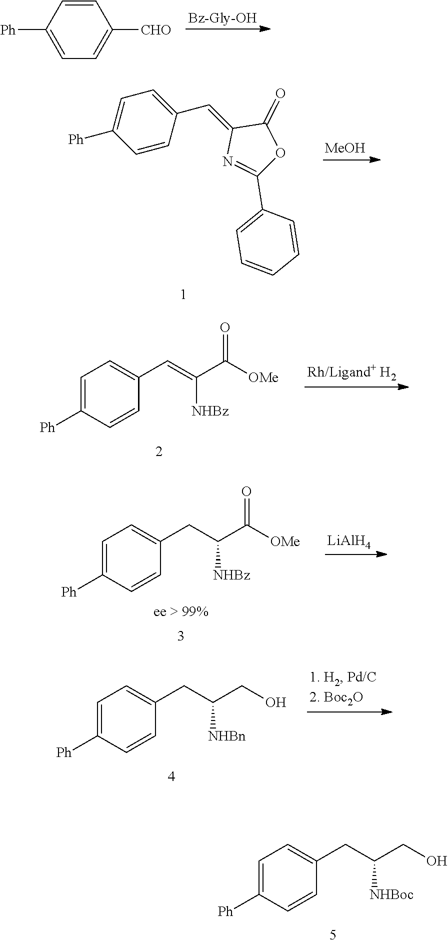

The process disclosed in WO 2014/032627 is depicted in the following scheme

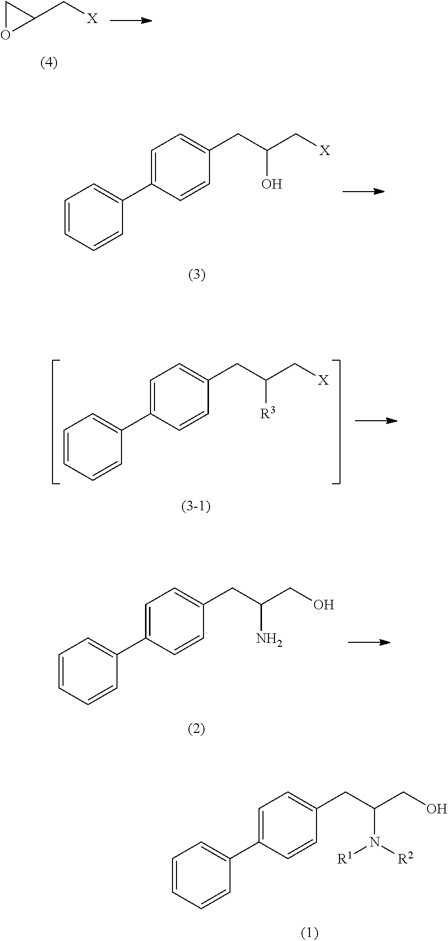

However, these processes still have disadvantages such as potentially dangerous reactants or use of expensive catalysts and/or limited stereo-selectivity. Therefore, there is still a need to design chemical processes to provide cheap ways to access said starting materials for the synthesis of sacubitril which are suitable for industrial scale production under economically and environmentally favorable conditions and provide such drug substance precursors in high chemical purity and with high stereo-chemical selectivity.

### SUMMARY OF THE INVENTION

The invention relates to novel intermediates and process steps and processes for the manufacture of a compound of formula (II), especially (II-a) represented below, and its further use in the manufacture of sacubitril.

In a first aspect, the present invention relates to the novel compound of formula (II), or a salt thereof

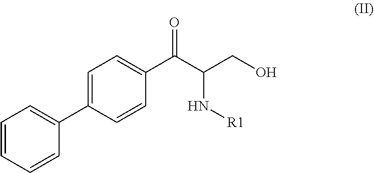

preferably a compound of formula (II-a), or a salt thereof

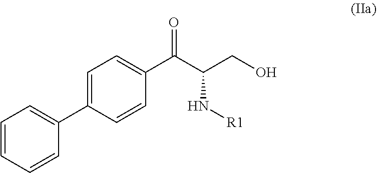

wherein R1 is hydrogen or a nitrogen protecting group.

In a second aspect, the present invention relates to a process for preparing such a novel compound of formula (II), or a salt thereof

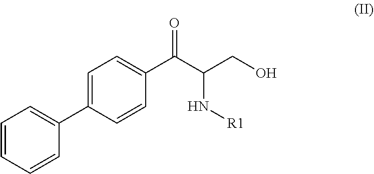

preferably the compound of formula (II-a), or a salt thereof

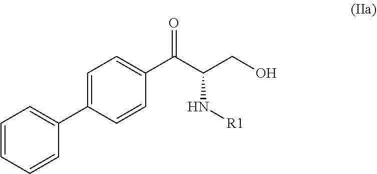

wherein R1 is hydrogen or a nitrogen protecting group,
by a process comprising reacting a compound of formula (III), or a salt thereof,

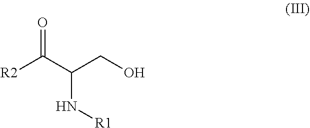

preferably of formula (III-a), or a salt thereof,

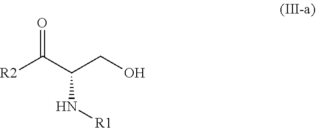

wherein R1 is hydrogen or a nitrogen protecting group, and R2 is a CO-activating group,
with a biphenylic compound.

In a third aspect, the invention relates to processes using the novel compound of formula (II).

One aspect relates to a process for producing a free or preferably nitrogen-protected amino alcohol compound according to formula (I), or a salt thereof,

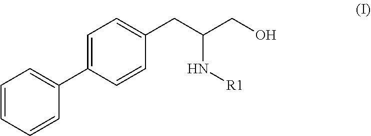

preferably a compound of formula (I-a), or a salt thereof

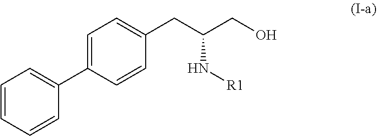

wherein R1 is hydrogen or a nitrogen protecting group,
said process comprising reacting the novel compound of formula (II), or a salt thereof

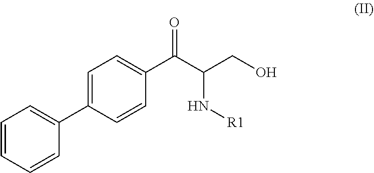

preferably a compound of formula (II-a), or a salt thereof

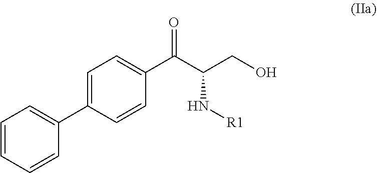

wherein R1 is hydrogen or a nitrogen protecting group,
by reduction of its benzylic keto group.

As indicated, the compound of formula (II) represents a key intermediate for the synthesis of NEP inhibitors or prodrugs thereof, or salts thereof. Accordingly, in a fourth aspect, the present invention relates to the use of the novel compound of formula (II), or a salt thereof

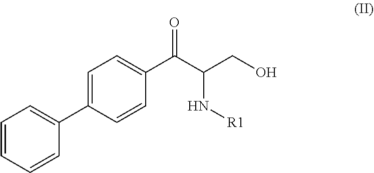

preferably a compound of formula (II-a), or a salt thereof

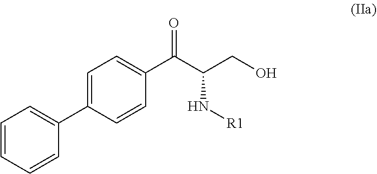

wherein R1 is hydrogen or a nitrogen protecting group,
in the manufacture of a compound of formula (X)

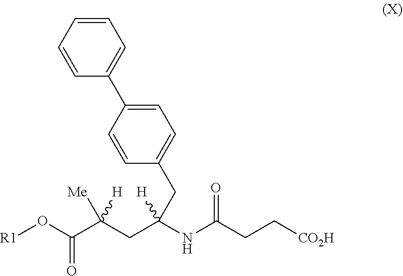

preferably of formula (X-a)

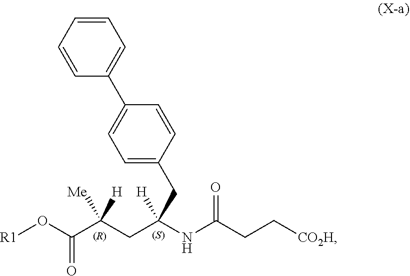

wherein R1 is hydrogen or C₁-C₆-alkyl, preferably ethyl,
preferably in the manufacture of the NEP-inhibitor N-(3-carboxy-1-oxopropyl)-(4S)-(p-phenylphenylmethyl)-4-amino-(2R)-methyl butanoic acid, or a salt thereof, or the NEP-inhibitor pro-drug N-(3-carboxy-1-oxopropyl)-(4S)-(p-phenylphenylmethyl)-4-amino-(2R)-methyl butanoic acid ethyl ester (known in the art as AHU377 or sacubitril) or a salt thereof.

In further embodiments, the invention relates to any one or more of the novel compounds, processes and catalysts represented in the claims, which are incorporated here by reference.

The invention also relates to any sequential combination of the process steps described above and below.

In its above mentioned aspects which are also given in more detail below the present invention provides the following advantages: The synthesis route is suitable for industrial scale processing. The synthesis route is economically and environmentally favorable. The compounds of formula (I) and (XVI) which are intermediates desired for the synthesis of sacubitril can be produced with high yield and high stereoselectivity.

### DETAILED DESCRIPTION OF THE INVENTION

### General Terms

The general definitions used above and below, unless defined differently, have the following meanings, where replacement of one or more or all expressions or symbols by the more specific definitions can be made independently for each invention embodiment and lead to more preferred embodiments.

Where the plural form is used for compounds, starting materials, intermediates, salts, pharmaceutical preparations, diseases, disorders and the like, this intends to mean one (preferred) or more single compound(s), salt(s), pharmaceutical preparation(s), disease(s), disorder(s) or the like, where the singular or the indefinite article (“a”, “an”) is used, this does not intend to exclude the plural, but only preferably means “one”.

### Chiral Compounds

The term “chiral” refers to molecules which have the property of non-superimposability on their mirror image partner, while the term “achiral” refers to molecules which are superimposable on their mirror image partner.

In the formulae of the present application the term “ [symbol] ” on a C-sp³ represents a covalent bond, wherein the stereochemistry of the bond is not defined. This means that the term “ [symbol] ” on a C-sp³ comprises an (S) configuration as well as an (R) configuration of the respective chiral centre. Furthermore, mixtures, e.g. mixtures of enantiomers such as racemates, are also encompassed by the present invention. Especially preferred are single stereoisomers of the compounds of the formula (1) or (2), especially the specific ones of formula (1-a) and (1-b).

In the formulae of the present application the term “ [symbol] ” on a C-sp² represents a covalent bond, wherein the stereochemistry or the geometry of the bond is not defined. This means that the term “ [symbol] ” on a C-sp² comprises a (Z) configuration as well as a (E) configuration of the respective double bond. Furthermore, mixtures, e.g., mixtures of double bond isomers are also encompassed by the present invention.

In the formulae of the present application the term “ [symbol] ” on a C-sp³ indicates the absolute stereochemistry, either (R) or (S).

In the formulae of the present application the term “ [symbol] ” on a C-sp³ indicates the absolute stereochemistry, either (R) or (S).

In the formulae of the present application, the term “ [symbol] ” indicates a C-sp³-C-sp³ bond or a C-sp²-C-sp² bond.

The compounds of the present invention can possess one or more asymmetric centers. The preferred absolute configurations are as indicated herein specifically. However, any possible pure enantiomer, pure diastereoisomer, or mixtures thereof, e.g., mixtures of enantiomers, such as racemates, are encompassed by the present invention.

Compounds with a stereogenic center but without indication of a specific configuration are considered mixtures of compounds with the respective configurations, e.g. R,R; R,S; S,R and SS, or pure enantiomers/diastereomers.

Stereoisomeric, especially enantiomeric, purity, is where mentioned referring to all diastereomers of the compound taken together (100%). It is determined by chiral chromatography (examples include HPLC, uPLC and GC) or NMR (with addition of chiral entities and or metals).

The term “substantially optically pure” compound, as defined herein, refers to a compound obtained by a process according to the invention wherein the compound has an optical purity of at least 70% (ee=enantiomeric excess), more preferably of at least 90% (ee) and most preferably at least 95% (ee) or more, such as 100% (ee).

### Prodrugs

The term “pro-drug”, as used herein, represents in particular compounds which are transformed in vivo to the parent compound, for example, by hydrolysis in blood, for example as described in T. Higuchi and V. Stella, “Pro-drugs as Novel Delivery Systems”, volume 14 of the ACS Symposium Series; Edward B. Roche, editor, “Bioreversible Carriers in Drug Design”, American Pharmaceutical Association and Pergamon Press, 1987; H Bundgaard, editor, “Design of Prodrugs”, Elsevier, 1985; Judkins et al. Synthetic Communications 1996, 26, 4351-4367, and “The Organic Chemistry of Drug Design and Drug Action”, second edition, R. B. Silverman (particularly chapter 8, pages 497-557), Elsevier Academic Press, 2004.

Pro-drugs therefore include drugs having a functional group which has been transformed into a reversible derivative thereof. Typically, such prodrugs are transformed to the active drug by hydrolysis. As examples may be mentioned the following:

| Functional Group | Reversible derivative |
|---|---|
| Carboxylic acid | Esters, including e.g. alkyl esters |
| Alcohol | Esters, including e.g. sulfates and |
| | phosphates as well as carboxylic acid esters |
| Amine | Amides, carbamates, imines, enamines, |
| Carbonyl (aldehyde, | Imines, oximes, acetals/ketals, enol esters, |
| ketone) | oxazolidines and thiazoxolidines |

Pro-drugs also include compounds convertible to the active drug by an oxidative or reductive reaction. As examples may be mentioned:

Oxidative Activation
N- and O-dealkylation
Oxidative deamination
N-oxidation
Epoxidation

Reductive Activation
Azo reduction
Sulfoxide reduction
Disulfide reduction
Bioreductive alkylation
Nitro reduction

### NEP Inhibitor

The term “NEP inhibitor” describes a compound which inhibits the activity of the enzyme neutral endopeptidase (NEP, EC 3.4.24.11).

In the present invention the terms “NEP-inhibitor” or “NEP-inhibitor prodrug” relates to the substances as such or to salts thereof, preferably pharmaceutically acceptable salts thereof. Examples are sodium, potassium, magnesium, calcium or ammonium salts. Calcium salts are preferred.

The NEP inhibitor prodrug N-(3-carboxy-1-oxopropyl)-(4S)-p-phenylphenylmethyl)-4-amino-(2R)-methylbutanoic acid ethyl ester optionally may be further reacted to obtain the active NEP inhibitor N-(3-carboxy-1-oxopropyl)-(4S)-p-phenylphenylmethyl)-4-amino-(2R)-methylbutanoic acid, either in vitro or in vivo.

### Substituent Definitions

Alkyl is defined as a radical or part of a radical as a straight or branch (one or, if desired and possible, more times) carbon chain, and is especially C₁-C₇-alkyl, preferably C₁-C₆-alkyl, more preferably C₁-C₄-alkyl.

The terms “C₁-C₇-”, “C₁-C₆-” and “C₁-C₄-”, respectively, define a moiety with up to and including maximally 7, especially up to and including maximally 6 and 4 respectively, carbon atoms, said moiety being branched (one or more times) or straight-chained and bound via a terminal or a non-terminal carbon.

Cycloalkyl is, for example, C₃-C₇-cycloalkyl and is, for example, cyclopropyl, cyclobutyl, cyclopentyl, cyclohexyl and cycloheptyl. Cyclopentyl and cyclohexyl are preferred.

Alkoxy is, for example, C₁-C₇-alkoxy and is, for example, methoxy, ethoxy, n-propyloxy, isopropyloxy, n-butyloxy, isobutyloxy, sec-butyloxy, tert-butyloxy and also includes corresponding pentyloxy, hexyloxy and heptyloxy radicals. C₁-C₄-alkoxy is preferred.

Alkanoyl is, for example, C₂-C₈-alkanoyl and is, for example, acetyl [—C(═O)Me], propionyl, butyryl, isobutyryl or pivaloyl. C₂-C₅-Alkanoyl is preferred, especially acetyl.

Halo or halogen is preferably fluoro, chloro, bromo or iodo, most preferably, chloro, bromo, or iodo.

Halo-alkyl is, for example, halo-C₁-C₇-alkyl and is in particular halo-C₁-C₄-alkyl, such as trifluoromethyl, 1,1,2-trifluoro-2-chloroethyl or chloromethyl. Preferred halo-C₁-C₇-alkyl is trifluoromethyl.

Alkenyl may be linear or branched alkyl containing a double bond and comprising preferably 2 to 12 carbon atoms, 2 to 10 carbon atoms being especially preferred. Particularly preferred is a linear C₂-C₇-alkenyl, more preferably C₂-C₄-alkenyl. Some examples of alkyl groups are ethyl and the isomers of propyl, butyl, pentyl, hexyl, heptyl, octyl, nonyl, decyl, undecyl, dodecyl, tetradecyl, hexadecyl, octacyl and eicosyl, each of which containing a double bond. Especially preferred is allyl.

Alkylene is a bivalent radical derived from C₁₋₇-alkyl and is especially C₂-C₇-alkylene or C₂-C₇-alkylene and, optionally, can be interrupted by one or more, e.g. up to three oxygen, NR14 or sulfur, wherein R14 is alkyl, each of which can be unsubstituted or substituted, by one or more substituents independently selected from for example, C₁-C₇-alkyl, C₁-C₇-alkoxy-C₁-C₇-alkyl or C₁-C₇-alkoxy.

Alkenylene is a bivalent radical derived from C₂₋₇-alkenyl and can be interrupted by one or more, e.g. up to three oxygen, NR14 or sulfur, wherein R14 is alkyl, and is unsubstituted or substituted by one or more, e.g. up to three substitutents, preferably independently selected from the substituents mentioned above for alkylene.

Aryl being a radical or part of a radical is, for example C₆₋₁₀-aryl, and is preferably a mono- or polycyclic, especially monocyclic, bicyclic or tricyclic aryl moiety with 6 to 10 carbon atoms, such as phenyl, naphthyl or fluorenyl preferably phenyl, and which can be unsubstituted or substituted, by one or more substituents, independently selected from, e.g. C₁-C₇-alkyl, C₁-C₇-alkoxy-C₁-C₇-alkyl or C₁-C₇-alkoxy.

The term arylalkyl refers to aryl-C₁-C₇-alkyl, wherein aryl is as defined herein and is for example benzyl.

The term carboxyl refers to —CO₂H.

Aryloxy refers to an aryl-O— wherein aryl is as defined above.

Unsubstituted or substituted heterocyclyl is a mono- or polycyclic, preferably a mono-, bi- or tricyclic-, most preferably mono-, unsaturated, partially saturated, saturated or aromatic ring system with preferably 3 to 14 (more preferably 5 to 14) ring atoms and with one or more, preferably one to four, heteroatoms, independently selected from nitrogen, oxygen, sulfur, S(═O)— or S—(=O)₂, and is unsubstituted or substituted by one or more, e.g. up to three substitutents, preferably independently selected from the group consisting of halo, C₁-C₇-alkyl, halo-C₁-C₇-alkyl, C₁-C₇-alkoxy, halo-C₁-C₇-alkoxy, such as trifluoromethoxy and C₁-C₇-alkoxy-C₁-C₇-alkoxy. When the heterocyclyl is an aromatic ring system, it is also referred to as heteroaryl. Heterocyclyl is preferably imizazolyl, pyrazolyl, oxazolyl, isoxazolyl, thiazolyl, isothiazolyl, pyridinyl, pyranyl, diazionyl, oxazinyl, thiazinyl, dioxinyl, dithiinyl, azepanyl, oxepanyl, thiepanyl, indolyl, isoindoly, quinolinyl, isoquinolinyl, benzazepinyl, carbazolyl, imidazolidinyl, pyrazolidinyl, oxazolidinyl, isoxazolinidyl, thiazolidy, dioxolanyl, dithiolanyl, furazanyl, oxadiazolyl, thiadiazolyl, dithiazolyl, tetrazolyl, piperidinyl, piperazinyl, morpholinyl, thiomorpholinyl, oxothiomorpholinyl, dioxothiomorpholinyl, dioxanyl, dithianyl, azepanyl, oxepanyl, thiepanyl, or benzo-fused variants thereof.

In heterocyclylalkyl, the heterocyclyl is preferably as just defined and is attached to an alkyl as defined for alkyl. Examples are imidazolylmethyl, pyridylmethyl or piperidinylmethyl.

Acetyl is —C(═O)C₁-C₇-alkyl, preferably —C(═O)Me.

Sulfonyl is (unsubstituted or substituted) C₁-C₇-alkylsulfonyl, such as methylsulfonyl, (unsubstituted or substituted) phenyl- or naphthyl-C₁-C₇-alkylsulfonyl, such as phenylmethanesulfonyl, or (unsubstituted or substituted) phenyl- or naphthyl-sulfonyl; wherein if more than one substituent is present, e.g. one to three substitutents, the substituents are selected independently from cyano, halo, halo-C₁-C₇-alkyl, halo-C₁-C₇-alkyloxy- and C₁-C₇-alkyloxy. Especially preferred is C₁-C₇-alkylsulfonyl, such as methylsulfonyl, and (phenyl- or naphthyl)-C₁-C₇-alkylsulfonyl, such as phenylmethanesulfonyl.

Sulfenyl is (unsubstituted or substituted) C₆₋₁₀-aryl-C₁-C₇-alkylsulfenyl or (unsubstituted or substituted) C₆₋₁₀-arylsulfenyl, wherein if more than one substituent is present, e.g. one to four substitutents, the substituents are selected independently from nitro, halo, halo-C₁-C₇-alkyl and C₁-C₇-alkyloxy.

Imide refers to a (unsubstituted or substituted) functional group consisting of two acyl groups bound to nitrogen, preferably a cyclic group derived from dicarboxylic acids. Especially preferred is succinimidyl derived from succinic acid or phthalimidyl derived from phthalic acid. The imidyl group may be substituted by one or more substituents independently selected from for example, C₁-C₇-alkyl, C₁-C₇-alkoxy-C₁-C₇-alkyl, C₁-C₇-alkoxy or halo.

Azide refers to a group —N═N+=N⁻.

Silyl, as used herein, refers to a group according to the formula —SiR11R12R13, wherein R11, R12 and R13 are, independently of each other, C₁-C₇-alkyl, C₆-C₁₀-aryl or phenyl-C₁-C₄-alkyl. Preferred examples for R11, R12 and R13 are methyl, ethyl, isopropyl, tert-butyl, phenyl or phenyl-C₁₋₄-alkyl.

### Salts

Salts are especially pharmaceutically acceptable salts or generally salts of any of the intermediates mentioned herein, except if salts are excluded for chemical reasons the skilled person will readily understand. They can be formed where salt forming groups, such as basic or acidic groups, are present that can exist in dissociated form at least partially, e.g. in a pH range from 4 to 10 in aqueous solutions, or can be isolated especially in solid, especially crystalline, form.

Such salts are formed, for example, as acid addition salts, preferably with organic or inorganic acids, from compounds or any of the intermediates mentioned herein with a basic nitrogen atom (e.g. imino or amino), especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, lactic acid, fumaric acid, succinic acid, citric acid, amino acids, such as glutamic acid or aspartic acid, maleic acid, hydroxymaleic acid, methylmaleic acid, benzoic acid, methane- or ethane-sulfonic acid, ethane-1,2-disulfonic acid, benzenesulfonic acid, 2-naphthalenesulfonic acid, 1,5-naphthalene-disulfonic acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acid group are present in the same molecule, any of the intermediates mentioned herein may also form internal salts.

For isolation or purification purposes of any of the intermediates mentioned herein it is also possible to use pharmaceutically unacceptable salts, for example picrates or perchlorates.

In view of the close relationship between the compounds and intermediates in free form and in the form of their salts, including those salts that can be used as intermediates, for example in the purification or identification of the compounds or salts thereof, any reference to “compounds”, “starting materials” and “intermediates” hereinbefore and hereinafter is to be understood as referring also to one or more salts thereof or a mixture of a corresponding free compound, intermediate or starting material and one or more salts thereof, each of which is intended to include also any solvate or salt of any one or more of these, as appropriate and expedient and if not explicitly mentioned otherwise. Different crystal forms may be obtainable and then are also included.

### Nitrogen Protecting Groups

The term “nitrogen protecting group” comprises any group which is capable of reversibly protecting a nitrogen functionality, preferably an amine and/or amide functionality. Preferably the nitrogen protecting group is an amine protecting group and/or an amide protecting group. Suitable nitrogen protecting groups are conventionally used e.g. in peptide chemistry and are described e.g. in the relevant chapters of standard reference works such as J. F. W. McOmie, “Protective Groups in Organic Chemistry”, Plenum Press, London and New York 1973, in P. G. M. Wuts and T. W. Greene, “Greene's Protective Groups in Organic Synthesis”, fourth edition, Wiley, N.J., 2007, and “The Peptides”; volume 3 (editors: E. Gross and J. Meienhofer), Academic Press, London and New York 1981, and “Methoden der organischen Chemie” (Methods of Organic Chemistry), Houben Weyl, fourth edition, volume 15/I, Georg Thieme Verlag, Stuttgart 1974.

Preferred nitrogen protecting groups generally comprise: unsubstituted or substituted C₁-C₆-alkyl, preferably C₁-C₄-alkyl, more preferably C₁-C₂-alkyl, most preferably C₁-alkyl, unsubstituted or substituted C₂₋₄-alkenyl, wherein each C₁-C₆-alkyl and C₂₋₄-alkenyl is optionally mono-, di- or tri-substituted by trialkylsilyl-C₁-C₇-alkoxy (e.g. trimethylsilylethoxy), cycloalkyl, aryl, preferably phenyl, or a heterocyclic group, preferably pyrrolidinyl, wherein the cycloalkyl group, the aryl ring or the heterocyclic group is unsubstituted or substituted by one or more, e.g. two or three residues, e.g. selected from the group consisting of C₁-C₇-alkyl, hydroxy, C₁-C₇-alkoxy, C₂-C₈-alkanoyl-oxy, halogen, nitro, cyano, and CF₃; aryl-C₁-C₂-alkoxycarbonyl (preferably phenyl-C₁-C₂-alkoxycarbonyl e.g. benzyloxycarbonyl); C₁₋₁₀-alkenyloxycarbonyl; C₁₋₆-alkylcarbonyl (e.g. acetyl or pivaloyl); C₆₋₁₀-arylcarbonyl; C₁₋₆-alkoxycarbonyl (e.g. tert-butoxycarbonyl); C₆₋₁₀-aryl-C₁₋₆-alkoxycarbonyl; allyl or cinnamyl; sulfonyl or sulfenyl; a succinimidyl group, substituted silyl, e.g. triarylsilyl or trialkylsilyl (e.g. triethylsilyl).

Examples of preferred nitrogen protecting groups are acetyl, benzyl, cumyl, benzhydryl, trityl, benzyloxycarbonyl (Cbz), 9-fluorenylmethyloxycarbony (Fmoc), benzyloxymethyl (BOM), pivaloyl-oxy-methyl (POM), trichloroethxoycarbonyl (Troc), 1-adamantyloxycarbonyl (Adoc), allyl, allyloxycarbonyl, trimethylsilyl, tert-butyl-dimethylsilyl (TBDMS), triethylsilyl (TES), triisopropylsilyl (TIPS), trimethylsilyethoxymethyl (SEM), tert-butoxycarbonyl (BOC), tert-butyl, 1-methyl-1,1-dimethylbenzyl, (phenyl)methylbenzene, pyrridinyl and pivaloyl. Most preferred nitrogen protecting groups are acetyl, benzyl, benzyloxycarbonyl (Cbz), triethylsilyl (TES), trimethylsilyethoxymethyl (SEM), tert-butoxycarbonyl (BOC), pyrrolidinylmethyl and pivaloyl.

Examples of more preferred nitrogen protecting groups are, pivaloyl, pyrrolidinylmethyl, t-butoxycarbonyl, benzyl and silyl groups, particularly silyl groups according to the formula SiR11R12R13 group, wherein R11, R12 and R13 are, independently of each other, C₁-C₇-alkyl, C₆-C₁₀-aryl or phenyl-C₁-C₄-alkyl. Preferred examples for R11, R12 and R13 are methyl, ethyl, isopropyl, t-butyl and phenyl.

Examples of most preferred nitrogen protecting groups are tert-butoxycarbonyl (BOC), benzoyl, styryl, 1-butenyl, benzyl, p-methoxybenzyl (PMB) and pyrrolidinylmethyl, in particular pivaloyl and tert-butoxycarbonyl (BOC).

In one embodiment the term nitrogen protecting group refers to a group which is selected from the group consisting of

C₁-C₆-alkyl, which is unsubstituted or mono-, di- or tri-substituted by tri-C₁-C₆-alkylsilylC₁-C₇-alkoxy, C₆-C₁₀-aryl, or a heterocyclic group being a mono-, bi- or tricyclic ring system with 5 to 14 ring atoms and 1 to 4 heteroatoms independently selected from N, O, S, S(O) or S(O)₂, wherein the aryl ring or the heterocyclic group is unsubstituted or substituted by one, two or three residues, selected from the group consisting of C₁-C₇-alkyl, hydroxyl, C₁-C₇-alkoxy, C₂-C₈-alkanoyl-oxy, halogen, nitro, cyano, and CF₃;

C₆-C₁₀-aryl-C₁-C₂-alkoxycarbonyl; C₁-C₀₀-alkenyloxycarbonyl; C₁-C₆-alkylcarbonyl; C₆-C₁₀-arylcarbonyl; C₁-C₆-alkoxycarbonyl; C₆-C₁₀-aryl-C₁-C₆-alkoxycarbonyl; allyl; cinnamyl; sulfonyl; sulfenyl; succinimidyl, and silyl, wherein each silyl group is a SiR11R12R13 group, wherein R11, R12 and R13 are, independently of each other, C₁-C₇-alkyl, C₆-C₁₀-aryl or phenyl-C₁-C₄-alkyl.

Generally, in the present application the term “nitrogen protecting group” comprises any group which is capable of reversibly protecting an amino functionality.

If an embodiment requires the removal of the nitrogen protecting group, as defined above, the removal usually can be carried out by using known methods. e.g. as described in the references cited above. Preferably, the nitrogen protecting group, as defined above, is removed by using acidic or basic conditions. Examples for acidic conditions are hydrochloric acid, trifluoroacetic acid, sulphuric acid. Examples of basic conditions are lithium hydroxide, sodium ethoxide. Nucleophiles such as sodium borohydride can be used. In the case of N-benzyl as amino protecting group it can be removed by hydrogenation or by the use of some suitable oxidizing agents, e.g. ceric ammonium nitrate (CAN) or 2,3-dichloro-5,6-dicyano-p-benzoquinone (DDQ).

### Embodiments

The following sections describes in more detail, as necessary the individual process steps as laid out above or as depicted in the claims.

In the first aspect, the present invention relates to a compound of formula (II), or a salt thereof

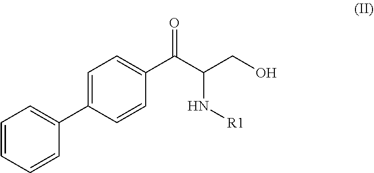

wherein R1 is hydrogen or a nitrogen protecting group.

In one embodiment thereof, the compound is of formula (II-a), or a salt thereof

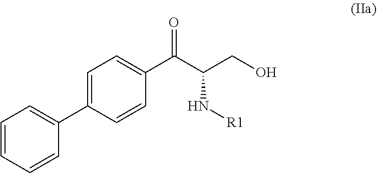

wherein R1 is hydrogen or a nitrogen protecting group.

In one embodiment, R1 is hydrogen.

In another embodiment, R1 is a nitrogen protecting group selected from C₁-C₆-alkyl, which is unsubstituted or mono-, di- or tri-substituted by tri-C₁-C₆-alkylsilylC₁-C₇-alkoxy, C₆-C₁₀-aryl, or a heterocyclic group being a mono-, bi- or tricyclic ring system with 5 to 14 ring atoms and 1 to 4 heteroatoms independently selected from N, O, S, S(O) or S(O)₂, wherein the aryl ring or the heterocyclic group is unsubstituted or substituted by one, two or three residues, selected from the group consisting of C₁-C₇-alkyl, hydroxyl, C₁-C₇-alkoxy, C₂-C₈-alkanoyl-oxy, halogen, nitro, cyano, and CF₃;

C₆-C₁₀-aryl-C₁-C₂-alkoxycarbonyl; C₁-C₁₀-alkenyloxycarbonyl; C₁-C₆-alkylcarbonyl; C₆-C₁₀-arylcarbonyl; C₁-C₆-alkoxycarbonyl; C₆-C₁₀-aryl-C₁-C₆-alkoxycarbonyl; allyl; cinnamyl; sulfonyl; sulfenyl; succinimidyl, and silyl,

wherein each silyl group is a SiR11R12R13 group, wherein R11, R12 and R13 are, independently of each other, C₁-C₇-alkyl, C₆-C₁₀-aryl or phenyl-C₁-C₄-alkyl.

In a preferred embodiment thereof, R1 is C₁-C₇-alkoxycarbonyl, especially tert-butoxycarbonyl (BOC).

In a second aspect, the present invention relates to a process for preparing such a novel compound of formula (II), or a salt thereof

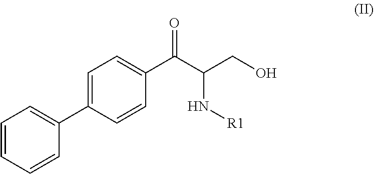

wherein R1 is hydrogen or a nitrogen protecting group,
by a process comprising reacting a compound of formula (III), or a salt thereof,

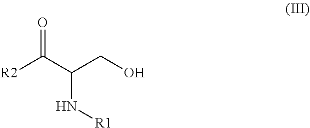

wherein R1 is hydrogen or a nitrogen protecting group, and R2 is a CO-activating group,
with a biphenylic compound.

In one embodiment thereof, the present invention relates to a process for preparing a compound of formula (II-a), or a salt thereof

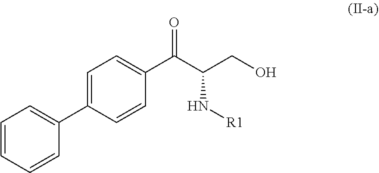

wherein R1 is hydrogen or a nitrogen protecting group,
by a process comprising reacting a compound of formula (III-a), or a salt thereof,

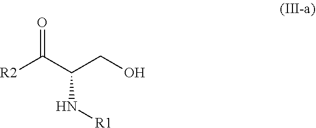

wherein R1 is hydrogen or a nitrogen protecting group, and R2 is a CO-activating group, with a biphenylic compound.

In compounds of formula (III) or (III-a) R2 is a CO-activating group. A suitable CO-activating group generally is any group, which can act as a leaving group. Examples of groups which can act as a CO-activating group are —NR₂, —OR, —SR or halogen, wherein R is hydrogen or (optionally substituted) alkyl or (optionally substituted) aryl.

Preferably, the following groups are suitable as CO-activating group R2 in compounds of formula (III) or (III-a):
(A) R2 can be an amino group, in particular, —NR12R13, wherein R12 and R13 are
   independently selected from the group consisting of alkyl, alkoxy, aryl, aryloxy, arylalkyl and arylalkoxy; preferably R12 is alkyl (eg methyl) and R13 is selected from the group consisting of alkoxy (eg. methoxy or ethoxy), aryloxy (eg. phenyloxy) and arylalkoxy (eg, benzyloxy); or
   together are unsubstituted or substituted alkylene or unsubstituted or substituted alkenylene; for example piperidinyl, morpholinyl, 1-alkylpiperazinyl (for example 1-methylpiperazinyl), 2-, 3-, 4-alkylpiperidinyl, 1,2,3,6-tetrahydropyridinyl, pyrrolidinyl or imidazolyl; or
   R12 is alkyl (eg. methyl) and R13 is —X—R14, wherein X is S and R14 is alkyl (eg. methyl or ethyl), aryl (eg. phenyl) or arylalkyl (eg. benzyl); or
   R12 is alkyl (eg. methyl) and R13 is —NRaRb, wherein Ra and Rb are independently selected from alkyl (eg. methyl or ethyl), aryl (eg. phenyl) or arylalkyl (eg. benzyl).

Preferred R2 is a dialkylated amino group, which can be cyclic (e.g. morpholinyl or imidazolyl) or acyclic (eg. dimethylamino). Cyclic amino groups preferably comprise a 5-member or 6-member ring, with or without additional substitution, in particular substitution refers to one or more substituents selected from the group consisting of halo, alkyl, alkoxy, aryl, aryloxy, arylalkyl and arylalkoxy. Also suitable are alkylaryl amino groups (e.g. phenylmethylamino) or diaryl amino groups (e.g. diphenylamino). Further suitable are so-called Weinreb derivatives (i.e. derivatives of methylmethoxyamine), in particular —NR12R13, wherein R12 is methyl or methoxy and R13 is independently selected from alkyl, alkoxy, aryl, aryloxy, arylalkyl or arylalkoxy. Further suitable are amino groups possessing an alkyl/aryl group and a coordinating group, e.g. alkoxy, alkylthio.
(B) R2 can be a group having the formula —X—R, wherein X is O or S and R is alkyl or aryl.

Furthermore, R4 can be a group having the formula —O—CO—R, wherein R is alkyl or aryl.
(C) R2 can be a halo, preferably chloro.
(D) R2 can be —O—R15 wherein R15 is —NR12R13, as defined above, or R15 is unsubstituted or substituted heterocyclyl.

Preferably, the CO-activating group is selected from N,O-dimethylhydroxylamino (N(Me)OMe), dimethylamino, morpholinyl, imidazolyl, —O-methyl, —O-ethyl, chloro, bromo, pivaloyl and acetyl, preferably N,O-dimethylhydroxylamino (N(Me)OMe) (Weinreb amide) or morpholinyl.

If the CO-activating group is chosen from the above groups (A) or (B) in formula (III), the residue R1 is preferably a nitrogen protecting group, as defined above, or alternatively hydrogen. If the CO-activating group is chosen from the above group (C) in formula (III), the residue R1 is preferably hydrogen.

The compound according to formula (III), or salt thereof, is reacted with a biphenylic compound.

In a preferred embodiment, the biphenylic compound can be activated. A suitable method for the activation is the preparation of an organometallic complex comprising a biphenyl ligand.

Preferred activated biphenylic compounds are biphenyl magnesium halide or di(biphenyl)magnesium (Grignard reagents). Suitable halides generally are chloride, bromide and iodide, wherein bromide is especially preferred. The Grignard reagent is either prepared according to appropriate reaction conditions well-known in the art or is commercially available (see e.g. Sigma-Aldrich, catalogue no. 562009, CAS no. 3315-91-1).

Further examples for activated biphenylic compounds are biphenyl lithium, biphenyl cuprate (low and higher-order cuprates) and biphenyl zinc. Those compounds can be used individually or in the presence of another metal, e.g. copper, zinc, palladium, platinum, iron, iridium or ruthenium.

In a preferred embodiment, the biphenylic compound is a biphenylic metal reagent, preferably a biphenyl magnesium halide, especially biphenyl magnesium bromide.

Generally, 2.0 to 2.5 equivalents of biphenylmagnesium halide or di(biphenyl)magnesium are used. In an embodiment initial deprotonation of the —OH group with, for example, another Grignard reagent (e.g. isopropylmagnesium chloride) or a base (e.g. sodium hydride) may be performed before addition of the activated biphenylic compound to reduce the required amount of biphenylmagnesium halide or di(biphenyl)magnesium. In this embodiment, 0.7 to 1.5 equivalents, preferably 1.0 to 1.25 equivalents are used.

Generally, there are two preferred embodiments to carry out the above-mentioned reaction:
1) Reacting a compound according to formula (III), or salt thereof, wherein R2 (the CO-activating group) is chosen from the above groups (A) or (B). In this case, an activated (e.g. metallated) biphenylic compound is preferably used, in particular a biphenyl magnesium halide is used. Then, the addition is preferably performed in the presence of a base, e.g. a Grignard reagent, such as isopropyl magnesium chloride or sec-butyl magnesium bromide, in an appropriate solvent, e.g. an ether, such as tetrahydrofuran, at preferred temperatures in the range from −78 to 20° C., e.g. from −20 to 0° C.
2) Reacting a compound according to formula (III), or salt thereof, wherein R2 (the CO-activating group) is chosen from the above group (C). In this case, biphenyl is preferably used as biphenylic compound. The reaction is preferably carried out in the presence of a suitable Lewis acid, e.g. aluminium trichloride. Alternatively, the biphenylic compound may be activated with a suitable functional group (for example para-silyl) to allow for milder conditions to be used during the Friedel-Crafts acylation. Furthermore, reference is made to the Friedel-Crafts method described in J. Am. Chem. Soc. 1981, 103, 6157.

Hence, it is preferred that R2 of formula (III) is N,O-dimethylhydroxylamino (N(Me)OMe) or morpholinyl and the biphenylic compound used is a biphenyl magnesium halide, preferably biphenyl magnesium bromide.

Alternatively, R2 of formula (III) is chloride and the biphenylic compound is biphenyl.

In one embodiment of the second invention aspect, the compound of formula (III) or a salt thereof,

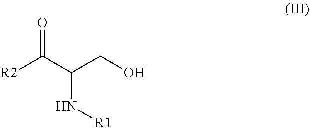

wherein R1 is hydrogen or a nitrogen protecting group, and R2 is a CO-activating group selected from N,O-dimethylhydroxylamino (N(Me)OMe) and morpholinyl,
can be obtained from a compound of the formula (IV)

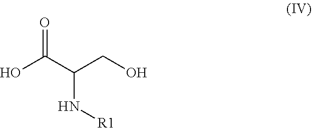

wherein R1 is hydrogen or a nitrogen protecting group,
by formation of the desired carboxylic acid amide with either N,O-dimethylhydroxylamine or a salt therefore, or morpholine or a salt thereof.

In one embodiment thereof, the compound of formula (III) is of formula (III-a)

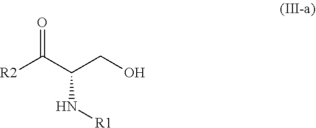

and the compound of formula (IV) is of formula (IV-a)

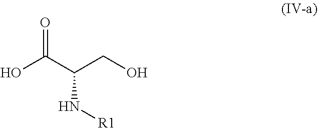

The reaction preferably uses a coupling agent, e.g. a diimide, such as 1-ethyl-3-(3-dimethylaminopropyl)carbodiimide hydrochloride (EDC), and either N,O-dimethylhydroxylamine or a salt therefore, or morpholine or a salt thereof, in an appropriate solvent, e.g. dichloromethane, at preferred temperatures in the range from −20 to 20° C., e.g. from −15 to 0° C. In case of use of the salts of N,O-dimethylhydroxylamine or morpholine, the reaction is carried out also in the presence of a base, e.g. an amine base, such as N-methylmorpholine,

The carboxylic acid compound of the formula (IV) is well known in the art and the enantiomer mixture and especially the L-form are commercially available (see e.g. Sigma-Aldrich, catalogue no. 15500, CAS no. 3262-72-4).

In a third aspect, the invention relates to processes using the novel compound of formula (II).

One aspect relates to a process for converting a compound of formula (II), or a salt thereof

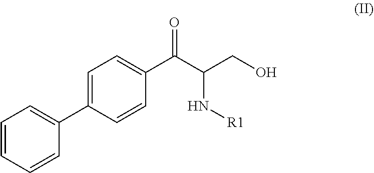

wherein R1 is hydrogen or a nitrogen protecting group,
into a compound according to formula (I), or a salt thereof

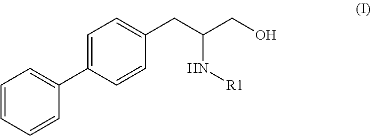

wherein R1 is hydrogen or a nitrogen protecting group,
said process comprising the reduction of the benzylic keto group of the compound of formula (II).

In one embodiment thereof, the compound of formula (II) is of formula (II-a),

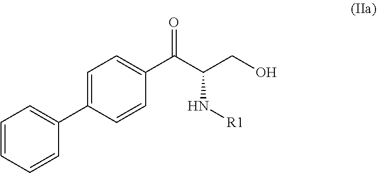

and the compound of formula (I) is of formula (I-a), or a salt thereof

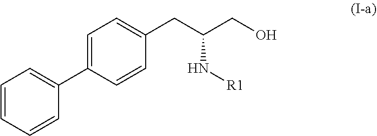

In one embodiment, the reduction is carried out under hydrogenation conditions, preferably by using hydrogen and palladium, in particular hydrogen on charcoal.

The reduction of the benzylic keto group occurs preferably by hydrogenation, usually with Pd catalysts (e.g. 10% Pd/C) in an appropriate solvent, e.g. an ester, such as ethyl acetate; a ketone, such as acetone; an ether, such as methyl tert-butyl ether; or an aromatic solvent, such as toluene; or a mixture of one of these solvents with acetic acid, optionally in the presence of additives, e.g. sodium acetate, sodium bicarbonate, triethylamine or copper sulfate, at preferred temperatures in the range from 10 to 50° C., e.g. from 20 to 40° C. and under preferred hydrogen pressures in the range from 1 to 20 bar, e.g. from 5 to 10 bar.

In a preferred embodiment, the Pd/C catalyst is Type 10R39 from Johnson Matthey (http://jmcct.com/products-services/product_p482.html).

In a further embodiment of this aspect, the compound of formula (II) or formula (II-a) is obtained by the processes described under the second aspect above, i.e. in one embodiment the present invention relates to a process for preparing a compound according to formula (I), or a salt thereof

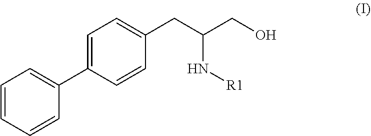

preferably a compound of formula (I-a), or a salt thereof

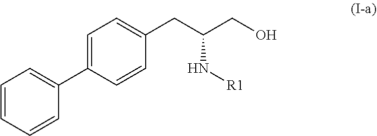

wherein R1 is hydrogen or a nitrogen protecting group,
by a process comprising
(i) reacting a compound of formula (III), or a salt thereof,

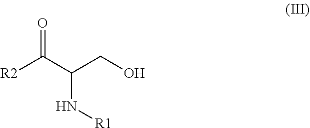

preferably of formula (III-a), or a salt thereof,

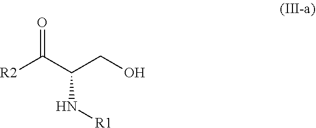

wherein R1 is hydrogen or a nitrogen protecting group, and R2 is a CO-activating group, with a biphenylic compound
to obtain a compound of formula (II), or a salt thereof

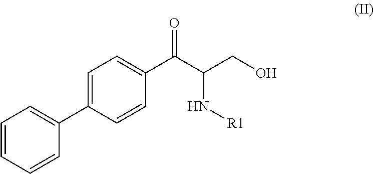

preferably a compound of formula (II-a), or a salt thereof

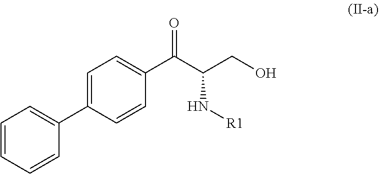

wherein R1 is hydrogen or a nitrogen protecting group, and
(ii) reacting the obtained compound of formula (II), or a salt thereof,
preferably the obtained compound of formula (II-a), or a salt thereof,
by a process comprising the reduction of the benzylic keto group of the compound of formula (II) to obtain the compound of formula (I).

In a further embodiment thereof, the present invention relates to a process for preparing a compound according to formula (I), or a salt thereof

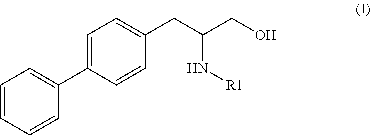

preferably a compound of formula (I-a), or a salt thereof

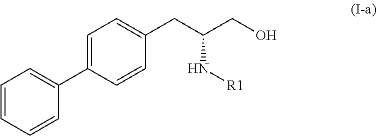

wherein R1 is hydrogen or a nitrogen protecting group,
by a process comprising
(i) reacting a compound of formula (IV), or a salt thereof,

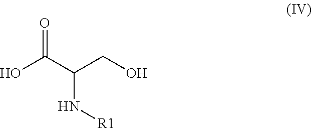

preferably of formula (IV-a), or a salt thereof,

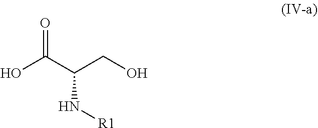

wherein R1 is hydrogen or a nitrogen protecting group,
with either N,O-dimethylhydroxylamine or a salt therefore, or morpholine, or a salt thereof to obtain a compound of formula (III), or a salt thereof,

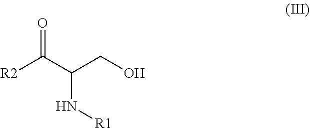

preferably of formula (III-a), or a salt thereof,

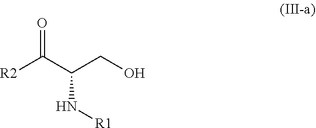

wherein R1 is hydrogen or a nitrogen protecting group, and R2 is a CO-activating group selected from N,O-dimethylhydroxylamino (N(Me)OMe) or morpholinyl,
(ii) reacting the obtained compound of formula (III) or a salt thereof, preferably the obtained compound of formula (III-a) or a salt thereof with a biphenylic compound
to obtain a compound of formula (II), or a salt thereof

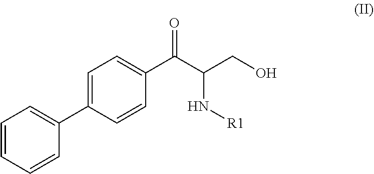

preferably a compound of formula (III-a), or a salt thereof

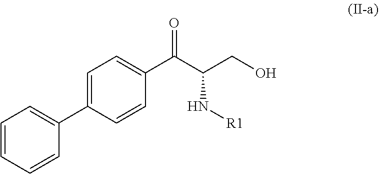

wherein R1 is hydrogen or a nitrogen protecting group, and
(iii) reacting the obtained compound of formula (II), or a salt thereof,
preferably the obtained compound of formula (III-a), or a salt thereof,
by a process comprising the reduction of the benzylic keto group of the compound of formula (II) to obtain the compound of formula (I).

In a further embodiment of the present invention, the obtained compound of formula (I), or a salt thereof,

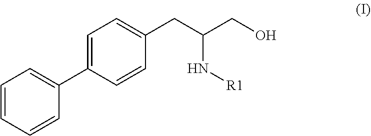

preferably a compound of formula (I-a), or a salt thereof

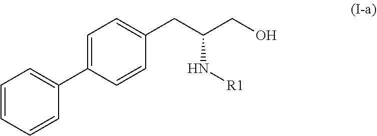

wherein R1 is hydrogen or a nitrogen protecting group,
is reacted by a process comprising a TEMPO mediated oxidation reaction or an oxidation with Dess-Martin periodinane to obtain a compound of formula (V), or a salt thereof,

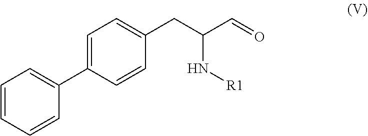

preferably a compound of formula (V-a), or a salt thereof

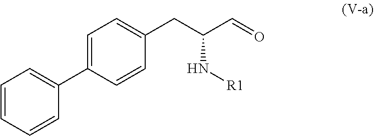

wherein R1 is hydrogen or a nitrogen protecting group.

Such reaction of the compound of formula (I), or more specifically of formula (I-a) to the corresponding aldehyde is performed by using a TEMPO mediated oxidation (see e.g. WO 2008/031567 or WO 2014/032627, page 24-25) or using alternative reaction conditions, such as oxidation with Dess-Martin periodinane (see e.g. WO 2008/136561).

In an embodiment relating to all previous embodiments of this invention, R1 in each formula where it is present is either hydrogen or a nitrogen protecting group selected from C₁-C₆-alkyl, which is unsubstituted or mono-, di- or tri-substituted by tri-C₁-C₆-alkylsilylC₁-C₇-alkoxy, C₆-C₁₀-aryl, or a heterocyclic group being a mono-, bi- or tricyclic ring system with 5 to 14 ring atoms and 1 to 4 heteroatoms independently selected from N, O, S, S(O) or S(O)₂, wherein the aryl ring or the heterocyclic group is unsubstituted or substituted by one, two or three residues, selected from the group consisting of C₁-C₇-alkyl, hydroxyl, C₁-C₇-alkoxy, C₂-C₈-alkanoyl-oxy, halogen, nitro, cyano, and CF₃;

C₆-C₁₀-aryl-C₁-C₂-alkoxycarbonyl; C₁-C₁₀-alkenyloxycarbonyl; C₁-C₆-alkylcarbonyl; C₆-C₁₀-arylcarbonyl; C₁-C₆-alkoxycarbonyl; C₆-C₁₀-aryl-C₁-C₆-alkoxycarbonyl; allyl; cinnamyl; sulfonyl; sulfenyl; succinimidyl, and silyl,
wherein each silyl group is a SiR11R12R13 group, wherein R11, R12 and R13 are, independently of each other, C₁-C₇-alkyl, C₆-C₁₀-aryl or phenyl-C₁-C₄-alkyl.

In a preferred embodiment, R1 in each formula where it is present is the nitrogen protecting group C₁-C₇-alkoxycarbonyl, preferably tert-butoxycarbonyl.

If required, any of the compounds (I), (11), (Ill), (IV) and (V) depicted without specific configuration at the amino group carrying carbon atom can be resolved into the corresponding pure enantiomer of the formula (I-a), (II-a), (III-a), (IV-a) or (V-a) by using customary methods for the resolution of enantiomers from enantiomer mixtures (such as racemates), e.g. by selective crystallization (e.g. via diastereomeric salts) from solutions or emulsions or chiral chromatography. Such methods are well-known in the art.

One embodiment of the present invention also relates to a process for preparing NEP inhibitor prodrug N-(3-carboxyl-1-oxopropyl)-(4S)-(p-phenylphenylmethyl)-4-amino-(2R)-methyl butanoic acid ethyl ester, or a salt thereof, comprising the manufacture of a compound of formula (I) or salt thereof, a compound of formula (II) or a salt thereof, or a compound of formula (V) or a salt thereof by any one of the processes as described in the aforementioned embodiments.

For example, in one embodiment of the present invention the synthesis of N-(3-carboxy-1-oxopropyl)-(4S)-p-phenylphenylmethyl)-4-amino-(2R)-methylbutanoic acid ethyl ester, or a salt thereof, starts from a compound of formula (I-a), or salt thereof, and a compound of formula (V-a), or a salt thereof, respectively. Preferably, said reaction comprises the following steps or steps in analogy thereto (see also WO 2008/031567 or WO 2014/032627, page 24-25):

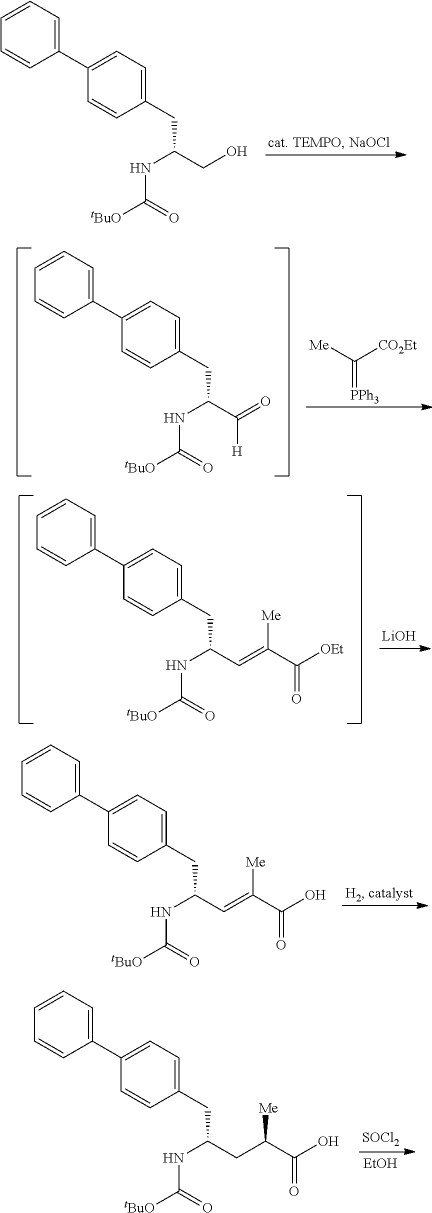

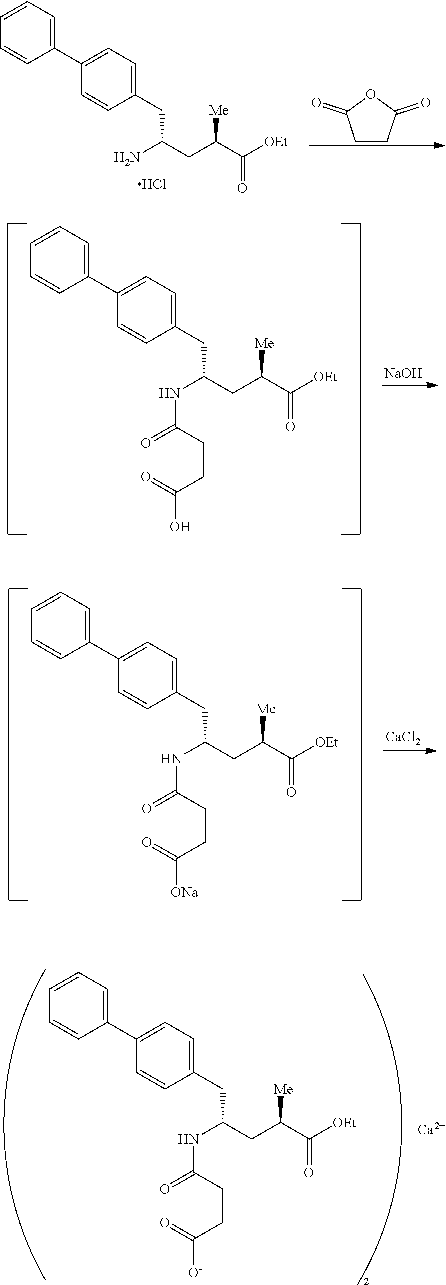

After the aforementioned TEMPO oxidation, the aldehyde of formula (V-a) is then subjected to a Wittig reaction with carbethoxyethylidene-triphenylphosphorane to deliver (R)-5-biphenyl-4-yl-4-tert-butoxycarbonylamino-2-methylpent-2-enoic acid ethyl ester. The ester or—after saponification of the ester—the corresponding free acid (R)-5-biphenyl-4-yl-4-tert-butoxycarbonylamino-2-methylpent-2-enoic acid is then hydrogenated in the presence of a catalyst, whilst preferably producing the preferred diastereoisomer with high selectivity. Deprotection of the nitrogen functionality, i.e. removal of the Boc group, if necessary re-introduction of the ethyl ester group, and subsequent coupling with succinic anhydride delivers the desired NEP inhibitor prodrug compound or a salt thereof. Optionally, the ester can be saponified to the free acid providing the NEP inhibitor drug compound.

In a final fourth aspect, the present invention relates to the use of a compound of formula (II), or a salt thereof

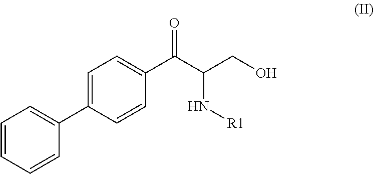

preferably a compound of formula (II-a), or a salt thereof

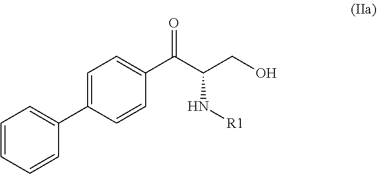

wherein R1 is hydrogen or a nitrogen protecting group,
in the synthesis of the NEP-inhibitor N-(3-carboxy-1-oxopropyl)-(4S)-(p-phenylphenylmethyl)-4-amino-(2R)-methylbutanoic acid or a salt thereof or the NEP-inhibitor pro-drug N-(3-carboxy-1-oxopropyl)-(4S)-(p-phenylphenylmethyl)-4-amino-(2R)-methylbutanoic acid ethyl ester or a salt thereof.

Embodiments of the invention can also be found in the claims, especially in the dependent claims, which are incorporated here by reference.

The following examples illustrate the invention without limiting its scope.

Abbreviations used:
Aq., aq. Aqueous
Ac acetyl
Bu butyl
CDI N,N-carbonyldiimidazole
Et ethyl
h hour(s)
Me methyl
min minute(s)
Ph phenyl

### Overview I

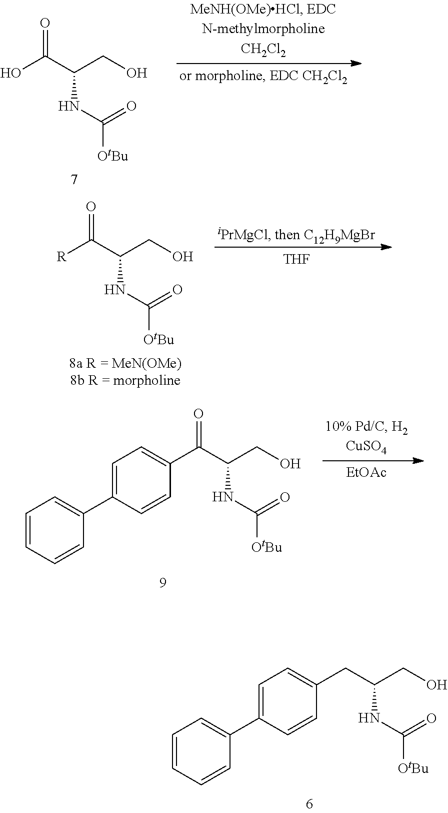

### Example 1a: Manufacture of (S)-tert-butyl (3-hydroxy-1-(methoxy(methyl)amino)-1-oxopropan-2-yl)carbamate 8a

To a suspension of Boc-L-serine (40.6 g, 197.8 mmol) in dichloromethane (800 mL) at −15° C. was added N,O-dimethylhydroxylamine hydrochloride (17.40 g, 198.8 mmol), followed by N-methylmorpholine (20.45 g, 202.2 mmol) and N-(3-dimethylaminopropyl)-N′-ethylcarbodiimide hydrochloride (EDC; 40.64 g, 207.7 mmol). The resulting clear, colorless solution was stirred at −15° C. for 1 h, then warmed to 0° C. and quenched by addition of 1 M aqueous HCl (150 mL). After warming to room temperature, the phases were separated and washed with water and saturated aqueous NaHCO₃ solution. The organic layer was dried over Na₂SO₄, filtered and concentrated under vacuum. The resulting white solid was dried at 45° C. under vacuum to give product 8a (39.11 g, 80% yield).

¹H-NMR (400 MHz, DMSO-d6): δ 1.37 (s, 9H), 3.10 (s, 3H), 3.40-3.51 (m, 1H), 3.51-3.61 (m, 1H), 3.71 (s, 3H), 4.50 (br. d, 1H), 4.81 (t, 1H), 6.76 (br. d, 1H); MS (ES-API): positive mode 249.0 [M+H]+.

### Example 1b: Manufacture of (S)-tert-butyl (3-hydroxy-1-morpholino-1-oxopropan-2-yl)carbamate 8b

To a suspension of Boc-L-serine (4.10 g, 19.98 mmol) in dichloromethane (36 mL) at −15° C. was added morpholine (1.92 g, 22.00 mmol), followed by N-(3-dimethylaminopropyl)-N′-ethylcarbodiimide hydrochloride (EDC; 3.22 g, 16.80 mmol). The resulting clear, colorless solution was stirred at −15° C. for 3 h, then quenched by addition of 1 M aqueous HCl (20 mL). The phases were separated, the aqueous layer was extracted with ethyl acetate, and the combined organic layers were washed with 30% aqueous KHCO₃ solution and water. The organic layer was dried over Na₂SO₄, filtered and concentrated under vacuum. Purification by chromatography (silica gel, diethyl ether) gave product 8b (1.64 g, 30% yield) as a clear, colorless oil.

¹H-NMR (400 MHz, DMSO-d6): δ 1.45 (s, 9H), 3.53-3.63 (m, 2H), 3.63-3.76 (m, 7H), 3.85 (dd, 1H), 4.55-4.66 (m, 1H), 5.67 (br. d, 1H); MS (ES-API): positive mode 275.3 [M+H]+.

### Example 2a: Manufacture of (S)-tert-butyl (1-([1,1′-biphenyl]-4-yl)-3-hydroxy-1-oxopropan-2-yl)carbamate 9 from (S)-tert-butyl (3-hydroxy-1-(methoxy(methyl)amino)-1-oxopropan-2-yl)carbamate 8a

To a solution of (S)-tert-butyl (3-hydroxy-1-(methoxy(methyl)amino)-1-oxopropan-2-yl)carbamate 8a (9.93 g, 40.00 mmol) in tetrahydrofuran (49 mL) at −18° C. was added a solution of sec-butylmagnesium chloride (2.0 M in diethylether, 40 mL, 80.00 mmol) at maximum −15° C. After stirring at −18° C. for 10 min, a solution of biphenylmagnesium bromide (0.5 M in tetrahydrofuran, 143 mL, 71.50 mmol) was added at maximum −15° C. The reaction mixture was warmed to 0° C. and stirred at this temperature for 1 h, then further warmed to room temperature and stirred at this temperature for another 3.5 h. After cooling to −15° C., the reaction mixture was quenched by addition of 1 M aqueous HCl (180 mL), then diluted with ethyl acetate. The organic layer was washed with water, and the water layers were back-extracted with ethyl acetate. The combined organic layers were dried over Na₂SO₄, filtered and concentrated under vacuum. Purification by chromatography (silica gel, heptanes/ethyl acetate) gave product 9 (6.53 g, 48% yield) as a white solid.

¹H-NMR (400 MHz, DMSO-d6): δ 1.37 (s, 9H), 3.66 (m, 1H), 3.72-3.82 (m, 1H), 4.87 (t, 1H), 5.09-5.19 (m, 1H), 7.05 (d, 1H), 7.40-7.47 (m, 1H), 7.47-7.56 (m, 2H), 7.72-7.79 (m, 2H), 7.84 (m, 2H), 8.07 (m, 2H); MS (ES-API): positive mode 364.3 [M+Na]⁺.

### Example 2b: Manufacture of (S)-tert-butyl (1-([1,1′-biphenyl]-4-yl)-3-hydroxy-1-oxopropan-2-yl)carbamate 9 from (S)-tert-butyl (3-hydroxy-1-morpholino-1-oxopropan-2-yl)carbamate 8b

To a solution of (S)-tert-butyl (3-hydroxy-1-morpholino-1-oxopropan-2-yl)carbamate 8b (1.21 g, 4.411 mmol) in tetrahydrofuran (10 mL) at −15° C. was added a solution of isopropylmagnesium chloride (1.0 M in tetrahydrofuran, 8.8 mL, 8.800 mmol) at maximum −12° C. After stirring at −15° C. for 5 min, a solution of biphenylmagnesium bromide (0.5 M in tetrahydrofuran, 13.2 mL, 6.600 mmol) was added at maximum −12° C. The reaction mixture was warmed to room temperature and stirred at this temperature for 5 h. After cooling to −10° C., the reaction mixture was quenched by addition of 1 M aqueous HCl (10 mL), then diluted with ethyl acetate and water. The organic layer was washed with water, and the water layers were back-extracted with ethyl acetate. The combined organic layers were dried over Na₂SO₄, filtered and concentrated under vacuum. Purification by chromatography (silica gel, diethyl ether) gave product 9 (0.75 g, 50% yield) as a white solid.

For analytical data, see preparation of compound 9 from compound 8a above.

### Example 3: Manufacture of (R)-tert-butyl (1-([1,1′-biphenyl]-4-yl)-3-hydroxypropan-2-yl)carbamate 6

A suspension of 10% Pd/C (type 10R39, Johnson Matthey; 40% dry weight loading, 30 mg dry weight, corrected for moisture of circa 50%) and (S)-tert-butyl (1-([1,1′-biphenyl]-4-yl)-3-hydroxy-1-oxopropan-2-yl)carbamate 9 in ethyl acetate (3 mL) was hydrogenated at 25° C. under hydrogen (3 bar pressure) for 18 h. The product 6 was isolated by filtration and purified by chromatography, if required.

¹H-NMR (400 MHz, DMSO-d6): δ 2.30 (d, 4H), 3.14 (m, 2H), 3.73 (s, 3H), 4.39 (t, 1H), 7.32 (m, 2H), 7.38 (m, 1H), 7.44-7.52 (m, 2H), 7.63-7.71 (m, 4H), 8.41 (br. s, 3H); MS (ES-API): positive mode 256.2 [M+H]⁺.

1. A compound of formula (II), or a salt thereof, 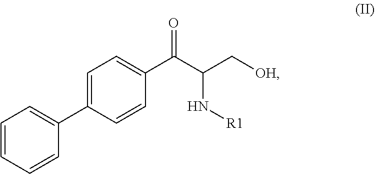
wherein R1 is hydrogen or C₁-C₇-alkoxycarbonyl.

2. The compound according to claim 1, wherein R1 is tert-butoxycarbonyl.

3. A process for preparing a compound of formula (II), or a salt thereof, 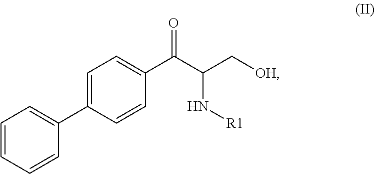
wherein R1 is hydrogen or a nitrogen protecting group selected from C₁-C₆-alkyl, which is unsubstituted or mono-, di- or tri-substituted by tri-C₁-C₆-alkylsilylC₁-C₇-alkoxy, C₆-C₁₀-aryl, or a heterocyclic group being a mono-, bi- or tricyclic ring system with 5 to 14 ring atoms and 1 to 4 heteroatoms independently selected from N, O, S, S(O) or S(O)₂, wherein the aryl ring or the heterocyclic group is unsubstituted or substituted by one, two or three residues, selected from the group consisting of C₁-C₇-alkyl, hydroxyl, C₁-C₇-alkoxy, C₂-C₈-alkanoyl-oxy, halogen, nitro, cyano, and CF₃; C₆-C₁₀-aryl-C₁-C₂-alkoxycarbonyl; C₁-C₁₀-alkenyloxycarbonyl; C₁-C₆-alkylcarbonyl; C₆-C₁₀-arylcarbonyl; C₁-C₇-alkoxycarbonyl; C₆-C₁₀-aryl-C₁-C₆-alkoxycarbonyl; allyl; cinnamyl; sulfonyl; sulfenyl; succinimidyl, and silyl, wherein each silyl group is a SiR11R12R13 group, and wherein R11, R12 and R13 are each independently C₁-C₇-alkyl, C₆-C₁₀-aryl, or phenyl-C₁-C₄-alkyl;
said process comprising reacting a compound of formula (III), or a salt thereof, 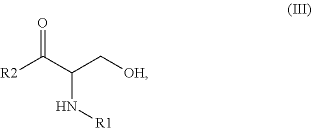
wherein R1 is hydrogen or a nitrogen protecting group selected from C₁-C₆-alkyl, which is unsubstituted or mono-, di- or tri-substituted by tri-C₁-C₆-alkylsilylC₁-C₇-alkoxy, C₆-C₁₀-aryl, or a heterocyclic group being a mono-, bi- or tricyclic ring system with 5 to 14 ring atoms and 1 to 4 heteroatoms independently selected from N, O, S, S(O) or S(O)₂, wherein the aryl ring or the heterocyclic group is unsubstituted or substituted by one, two or three residues, selected from the group consisting of C₁-C₇-alkyl, hydroxyl, C₁-C₇-alkoxy, C₂-C₈-alkanoyl-oxy, halogen, nitro, cyano, and CF₃; C₆-C₁₀-aryl-C₁-C₂-alkoxycarbonyl; C₁-C₁₀-alkenyloxycarbonyl; C₁-C₆-alkylcarbonyl; C₆-C₁₀-arylcarbonyl; C₁-C₇-alkoxycarbonyl; C₆-C₁₀-aryl-C₁-C₆-alkoxycarbonyl; allyl; cinnamyl; sulfonyl; sulfenyl; succinimidyl, and silyl, wherein each silyl group is a SiR11R12R13 group, and wherein R11, R12 and R13 are each independently C₁-C₇-alkyl, C₆-C₁₀-aryl, or phenyl-C₁-C₄-alkyl, and R2 is a CO-activating group,
with a biphenylic compound.

4. The process according to claim 3, wherein R2 is a CO-activating group selected from N,O-dimethylhydroxylamino (N(Me)OMe), dimethylamino, morpholinyl, imidazolyl, —O-methyl, —O-ethyl, chloro, bromo, pivaloyl and acetyl.

5. The process according to claim 3, wherein the biphenylic compound is a biphenylic metal reagent.

6. The process according to claim 3, wherein R2 is N,O-dimethylhydroxylamino (N(Me)OMe) or morpholinyl and the biphenylic compound is a biphenyl magnesium halide.

7. The process according to claim 3, wherein the compound of formula (III), or a salt thereof,
wherein R2 is a CO-activating group selected from N,O-dimethylhydroxylamino (N(Me)OMe) and morpholinyl, is prepared by a process which comprises reacting a compound of formula (IV), or a salt thereof, 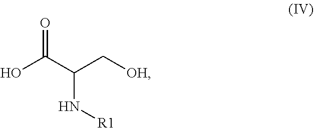
wherein R1 is hydrogen or a nitrogen protecting group selected from C₁-C₆-alkyl, which is unsubstituted or mono-, di- or tri-substituted by tri-C₁-C₆-alkylsilylC₁-C₇-alkoxy, C₆-C₁₀-aryl, or a heterocyclic group being a mono-, bi- or tricyclic ring system with 5 to 14 ring atoms and 1 to 4 heteroatoms independently selected from N, O, S, S(O) or S(O)₂, wherein the aryl ring or the heterocyclic group is unsubstituted or substituted by one, two or three residues, selected from the group consisting of C₁-C₇-alkyl, hydroxyl, C₁-C₇-alkoxy, C₂-C₈-alkanoyl-oxy, halogen, nitro, cyano, and CF₃; C₆-C₁₀-aryl-C₁-C₂-alkoxycarbonyl; C₁-C₁₀-alkenyloxycarbonyl; C₁-C₆-alkylcarbonyl; C₆-C₁₀-arylcarbonyl; C₁-C₇-alkoxycarbonyl; C₆-C₁₀-aryl-C₁-C₆-alkoxycarbonyl; allyl; cinnamyl; sulfonyl; sulfenyl; succinimidyl, and silyl, wherein each silyl group is a SiR11R12R13 group, and wherein R11, R12 and R13 are each independently C₁-C₇-alkyl, C₆-C₁₀-aryl, or phenyl-C₁-C₄-alkyl,
with either N,O-dimethylhydroxylamine, or a salt thereof, or morpholine, or a salt thereof, to form the desired carboxylic acid amide of formula (III).

8. A process for preparing a compound of formula (V), or a salt thereof, 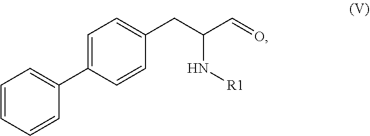
wherein R1 is hydrogen or a nitrogen protecting group selected from C₁-C₆-alkyl, which is unsubstituted or mono-, di- or tri-substituted by tri-C₁-C₆-alkylsilylC₁-C₇-alkoxy, C₆-C₁₀-aryl, or a heterocyclic group being a mono-, bi- or tricyclic ring system with 5 to 14 ring atoms and 1 to 4 heteroatoms independently selected from N, O, S, S(O) or S(O)₂, wherein the aryl ring or the heterocyclic group is unsubstituted or substituted by one, two or three residues, selected from the group consisting of C₁-C₇-alkyl, hydroxyl, C₁-C₇-alkoxy, C₂-C₈-alkanoyl-oxy, halogen, nitro, cyano, and CF₃; C₆-C₁₀-aryl-C₁-C₂-alkoxycarbonyl; C₁-C₁₀-alkenyloxycarbonyl; C₁-C₆-alkylcarbonyl; C₆-C₁₀-arylcarbonyl; C₁-C₇-alkoxycarbonyl; C₆-C₁₀-aryl-C₁-C₆-alkoxycarbonyl; allyl; cinnamyl; sulfonyl; sulfenyl; succinimidyl, and silyl, wherein each silyl group is a SiR11R12R13 group, and wherein R11, R12 and R13 are each independently C₁-C₇-alkyl, C₆-C₁₀-aryl, or phenyl-C₁-C₄-alkyl,
said process comprising,
(i) reducing the benzylic keto group of a compound of formula (II), or a salt thereof, 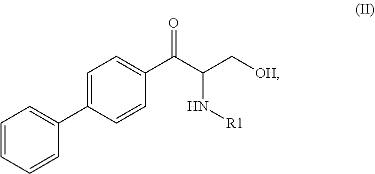
wherein R1 is hydrogen or a nitrogen protecting group selected from C₁-C₆-alkyl, which is unsubstituted or mono-, di- or tri-substituted by tri-C₁-C₆-alkylsilylC₁-C₇-alkoxy, C₆-C₁₀-aryl, or a heterocyclic group being a mono-, bi- or tricyclic ring system with 5 to 14 ring atoms and 1 to 4 heteroatoms independently selected from N, O, S, S(O) or S(O)₂, wherein the aryl ring or the heterocyclic group is unsubstituted or substituted by one, two or three residues, selected from the group consisting of C₁-C₇-alkyl hydroxyl, C₁-C₇-alkoxy, C₂-C₈-alkanoyl-oxy, halogen, nitro, cyano, and CF₃; C₆-C₁₀-aryl-C₁-C₂-alkoxycarbonyl; C₁-C₁₀-alkenyloxycarbonyl; C₁-C₆-alkylcarbonyl; C₆-C₁₀-arylcarbonyl; C₁-C₇-alkoxycarbonyl; C₆-C₁₀-aryl-C₁-C₆-alkoxycarbonyl; allyl; cinnamyl; sulfonyl; sulfenyl; succinimidyl, and silyl, wherein each silyl group is a SiR11R12R13 group, and wherein R11, R12 and R13 are each independently C₁-C₇-alkyl, C₆-C₁₀-aryl, or phenyl-C₁-C₄-alkyl,
to provide a compound according to formula (I), or a salt thereof, 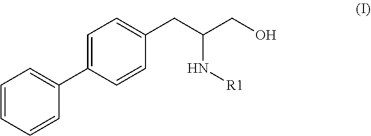
wherein R1 is hydrogen or a nitrogen protecting group selected from C₁-C₆-alkyl, which is unsubstituted or mono-, di- or tri-substituted by tri-C₁-C₆-alkylsilylC₁-C₇-alkoxy, C₆-C₁₀-aryl, or a heterocyclic group being a mono-, bi- or tricyclic ring system with 5 to 14 ring atoms and 1 to 4 heteroatoms independently selected from N, O, S, S(O) or S(O)₂, wherein the aryl ring or the heterocyclic group is unsubstituted or substituted by one, two or three residues, selected from the group consisting of C₁-C₇-alkyl, hydroxyl, C₁-C₇-alkoxy, C₂-C₈-alkanoyl-oxy, halogen, nitro, cyano, and CF₃; C₆-C₁₀-aryl-C₁-C₂-alkoxycarbonyl; C₁-C₁₀-alkenyloxycarbonyl; C₁-C₆-alkylcarbonyl; C₆-C₁₀-arylcarbonyl; C₁-C₇-alkoxycarbonyl; C₆-C₁₀-aryl-C₁-C₆-alkoxycarbonyl; allyl; cinnamyl; sulfonyl; sulfenyl; succinimidyl, and silyl, wherein each silyl group is a SiR11R12R13 group, and wherein R11, R12 and R13 are each independently C₁-C₇-alkyl, C₆-C₁₀-aryl, or phenyl-C₁-C₄-alkyl; and
(ii) oxidizing the compound of formula (I), or a salt thereof, through a TEMPO mediated oxidation reaction or using Dess-Martin periodinane to provide the aldehyde compound of formula (V).

9. The process according to claim 8, wherein the reduction of the benzylic keto group of the compound of formula (I) is carried out using hydrogenation.

10. The process according to claim 8, wherein the compound of formula (II), or a salt thereof, is prepared by the process according to claim 3.

11.-15. (canceled)

16. The compound according to claim 1, having a formula (II-a): 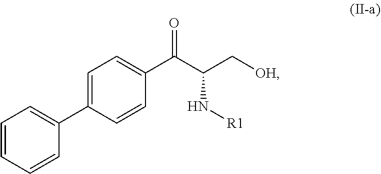
or a salt thereof.

17. The process according to claim 3, wherein the compound of Formula (II) has a formula (II-a): 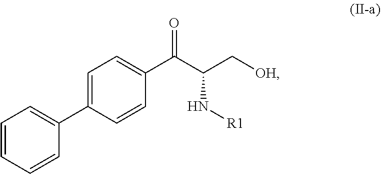
a salt thereof.

18. The process according to claim 3, wherein the compound of Formula (III) has a formula (III-a): 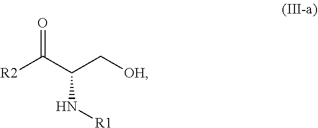
or a salt thereof.

19. The process according to claim 4, wherein R2 is a CO-activating group selected from N,O-dimethylhydroxylamino (N(Me)OMe) and morpholinyl.

20. The process according to claim 5, wherein the biphenylic compound is a biphenyl magnesium halide.

21. The process according to claim 20, wherein the biphenylic compound is biphenyl magnesium bromide.

22. The process according to claim 7, wherein the compound of Formula (V) has a Formula (IV-a): 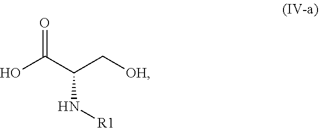
a salt thereof.

23. The process according to claim 8, wherein the compound of Formula (V) has a Formula (V-a): 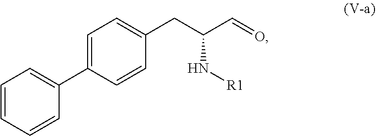
a salt thereof.

24. The process according to claim 8, wherein the compound of Formula (II) has a Formula (II-a): 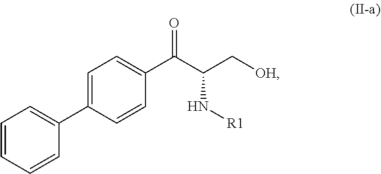
a salt thereof.

25. The process according to claim 8, wherein the compound of Formula (I) has a Formula (I-a): 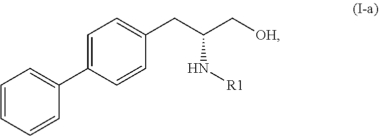
or a salt thereof.

26. The process according to claim 8, wherein the reduction of the benzylic keto group of the compound of formula (I) is carried out using hydrogen and palladium.

27. The process according to claim 8, wherein the reduction of the benzylic keto group of the compound of formula (I) is carried out using hydrogen and palladium on charcoal.
